(19) United States
(12) Patent Application Publication
(10) Pub. No.: US 2013/0041203 A1
(43) Pub. Date: Feb. 14, 2013

(54) BLOOD FLOW ASSIST DEVICES, SYSTEMS AND METHODS

(71) Applicants: MARLIN STEPHEN HEILMAN, SARVER, PA (US); DOUGLAS J. KOEBLER, IRWIN, PA (US); CHARLES ROBERT KOHLER, CHESWICK, PA (US); JON DAVID WAGNER, PITTSBURGH, PA (US); DAVID M. REILLY, PITTSBURGH, PA (US)

(72) Inventors: MARLIN STEPHEN HEILMAN, SARVER, PA (US); DOUGLAS J. KOEBLER, IRWIN, PA (US); CHARLES ROBERT KOHLER, CHESWICK, PA (US); JON DAVID WAGNER, PITTSBURGH, PA (US); DAVID M. REILLY, PITTSBURGH, PA (US)

(21) Appl. No.: 13/370,113

(22) Filed: Feb. 9, 2012

Related U.S. Application Data
(60) Provisional application No. 61/444,414, filed on Feb. 18, 2011.

(51) Int. Cl. A61M 1/12

(52) U.S. Cl. 600/16

(57) ABSTRACT

An implantable pump system for assisting blood flow includes a flexible conduit, at least one movable valve in fluid connection with the flexible conduit, a drive system comprising a rotary motor and a speed reducer operatively connected to the rotary motor; and a converter operatively connected to the drive system and operatively connected to the valve to drive the valve in a reciprocating linear manner. In a number of embodiments, the speed reducer includes a spur gear driving a ring gear, wherein the converter is operatively connected to the speed reducer.

### CROSS REFERENCE TO RELATED APPLICATION

This application claims benefit of U.S. Patent Application Ser. No. 61/444,414, filed Feb. 18, 2011, the disclosure of which is incorporated herein by reference.

### BACKGROUND

The following information is provided to assist the reader to understand the technology described below and certain environments in which such technology can be used. The terms used herein are not intended to be limited to any particular narrow interpretation unless clearly stated otherwise in this document. References set forth herein may facilitate understanding of the technology or the background thereof The disclosure of all references cited herein are incorporated by reference.

Heart failure, or the inability of the heart to pump sufficient blood for the body's needs, results in very poor quality of life, huge costs to society, and hundreds of thousands of yearly deaths. Heart failure is caused by an abnormally low cardiac output. Cardiac output is the outflow of blood from the heart and can be measured in liters of blood flow per minute or LPM. Cardiac output for a normal man at rest or during light activity is approximately 5 liters per minute. Severe heart failure exists when the cardiac output is between approximately 2.5 to 3.5 liters per minute. For an average man in heart failure having a heart rate of 80 beats per minute, the average amount of blood that is pumped with each heartbeat (sometimes referred to as stroke volume) might, for example, be 37 milliliters or ml. If the same man was not in heart failure, his heart might, for example, pump 62 milliliters with each heartbeat. An effective treatment for such heart failure would be to increase the low, 37 ml stroke volume up to the normal, 62 ml stroke volume.

The main pumping chamber of the heart or left ventricle or LV includes an inlet mitral valve and an outlet aortic valve. During left ventricular contraction or systole, the inlet valve closes as blood is pushed through the aortic valve into the aorta or main artery to the body. When the LV is resting during diastole, LV pressure may be between 2 and 20 mm of Hg pressure. This diastolic pressure is termed the LV preload. The preload will be in the higher end of its pressure range during heart failure. During active LV contraction or systole, the LV must eject its blood against the pressure in the aorta. Aortic pressure is typically between 70 and 140 mm Hg Pressure. This aortic pressure is termed the after-load. It is well known that, if the after-load is reduced in heart failure, the LV stroke volume will naturally increase and this increase is one reason that afterload-reducing drugs such as ACE-inhibitors help heart failure patients.

Blood pumps which lower the aortic pressure after-load can be desirable because such pumps allow the failing LV to eject more blood with less effort. However, no commercially available afterload reducing devices have thus far been shown to be practical for extended support of the failing LV. Instead, all long term (that is, months to years), commercially available heart assist devices, whether rotary turbine pumps or collapsing chamber pumps, go around or bypass the failing LV, pumping blood from the LV apex through the pump into the aorta. By doing so, those pumps act in parallel to the LV and compete with the LV in their pumping action. This pumping competition has several negative complications including right heart failure, fusion of the aortic valve over time and the risk of collapsing the LV. Collapsing chamber pumps are physically large and thus cannot be implanted in some small patients. Rotary turbine pumps are smaller, but have other limiting complications. For example, rotary turbine pumps induce high levels of shear stress in the blood elements and also may reduce the normal pulsatility of the blood entering the aorta. High shear stress on the blood cells promotes blood clotting which can lead to strokes and heart attacks. Physicians try to reduce this blood clotting by giving the patients anticoagulants, which, in turn, puts the patients at risk of excessive bleeding. These clotting and bleeding complications are substantial limitations to broader use of rotary turbine assist pumps.

For short-term heart assist (that is, hours to days), counterpulsation devices such as intraaortic balloon pumps or IABPs provide an afterload-reducing type of cardiac assist. See U.S. Pat. Nos. 4,733,652 and 3,692,018. The main benefit of such devices stems from after-load reduction of the left ventricle during systole and providing increased diastolic pressure for perfusing the coronary and other arteries during diastole. Typical patients needing this type of treatment suffer from cardiogenic shock or need perioperative circulatory support. The nature of IABP design restricts IABP to acute use only, since the bulky balloon drive mechanism remains outside the patient's body, necessitating patient confinement to a hospital bed.

U.S. Pat. No. 4,051,840 discloses a “dynamic aortic patch” which is surgically and permanently attached to the patient's descending aorta and is pneumatically activated by an external air pump. That pump lowers the LV after-load, facilitating left ventricular contraction and increasing stroke volume.

Pouch-type auxiliary ventricles attached to the patient's aorta have also been described. These devices use mechanical or pneumatic devices for pumping the blood contained in the pouch. See U.S. Pat. Nos. 3,553,736 and 4,034,742. Some of these devices have a single access port to the aorta that serves as both the inlet and the outlet for blood flow. Single port designs have the disadvantage of recirculation and relative flow stagnation, increasing the risk of clot formation and thromboembolism. Pouch-type auxiliary ventricles having both inlet and outlet ports to the aorta and are typically connected in parallel with the aorta. See, for example, U.S. Pat. Nos. 4,195,623 and 4,245,622.

U.S. Pat. Nos. 5,676,162, 5,676,651, and 5,722,930, disclose a single-stroke, moving valve pump designed for ascending aortic placement. That device uses a commercially available artificial heart valve with attached magnets and requires excision of a portion of the aorta. A series of separate electric coils step the valve/magnet combination forward in a sliding action within a cylinder. The device is quite large for the limited space available between the heart and the take-off vessels from the aorta to the upper body and brain. The device is designed to have one stroke in synchronization with each LV systole. The blood volume required for closing commercially available heart valves is typically 2-5 ml and therefore multiple smaller oscillations per heart contraction in such devices would suffer from volumetric inefficiency. Another problem with such devices is the tight crevice between the cylinder wall and the moving valve. This tight space results in high blood shear and the corresponding risk of stroke or blood clotting complications if anti-coagulant therapy is necessary. The same problem exists with a moving valve pump disclosed in U.S. Pat. No. 4,210,409, which included two valves (one stationary and one moving).

U.S. Pat. No. 5,147,281 discloses an oscillatory valve blood pump that is external to the body and fits in an enclosure the size of a briefcase. The pump uses a stationary coil to attract a magnetic tube encasing a one-way valve. A forward stroke of the one-way valve propels blood until the tube assembly stops and is repelled backward by return leaf springs that are charged during the forward stroke. A second stationary valve is sometimes in the circuit. A stretchable silicone rubber tube connects the tube or pipe-valve assembly with the pump inlet and outlet.

Nitta, S. et al., “The Newly Designed Univalved Artificial Heart,” ASAIO Transactions Vo. 37, No. 3, M240-M241 (1991) describes a “univalved artificial heart” powered electro-magnetically wherein the valve oscillates within a frequency range of 1 to 30 Hz. The valve is contained in a tube, with attached magnetic material. Stationary electric coils actuate the tube-magnet-valve combination. The valve is described as a jellyfish valve. A problem with jellyfish valves is the compound curvature or wrinkling of the membrane that occurs when the valve opens and closes. One can liken the action of the jellyfish valve to that of an umbrella that oscillates between a circular flat membrane and a wrinkled umbrella shape as it closes and opens. Wrinkling of the membrane is virtually impossible to prevent in a jellyfish valve and introduces stresses and strains that significantly limit the life of the valve.

U.S. Pat. No. 5,266,012 also uses a jellyfish valve in a vibrating pipe blood pump intended for use outside the body. Because the vibrating tube pump portion is separable from the drive mechanism. the blood-contacting portion of the pump is disposable.

U.S. Pat. No 7,588,530, describes a moving valve pump having a curved blood flow path as well as a moving valve pump having a linear blood flow path. U.S. Pat. No 7,588,530 discloses various drive mechanisms to oscillate the moving valve in synchronization with the R wave of the patient's electrocardiogram. In the case of a pump having a linear blood flow path, a linear motor is disclosed to drive the moving valve thereof.

Numerous pharmacologic, biologic, and mechanical interventions have been devised to address heart disease/failure. Nonetheless, heart failure remains a major public health problem with an estimated five million victims in the United States alone.

### SUMMARY

In one aspect an implantable pump system for assisting blood flow includes a flexible conduit, at least one movable valve in fluid connection with the flexible conduit, a drive system comprising a rotary motor and a speed reducer operatively connected to the rotary motor; and a converter operatively connected to the drive system and operatively connected to the valve to drive the valve in a reciprocating linear manner. In a number of embodiments, the speed reducer includes a spur gear driving a ring gear, wherein the converter is operatively connected to the speed reducer. The ring gear may, for example, be in operative connection with the converter. In a number of embodiments, the converter includes an eccentric member extending from the ring gear. In a number of embodiments, the converter further includes a rotating element connected to the eccentric member that engages a cam member operatively connected to the valve to drive the valve in a reciprocating, linear manner.

In a number of embodiments, any moving mechanical linkage of the drive system and between the drive system and the valve includes at least one rolling element bearing resistant to corrosion by a salt solution. The rolling element bearing may, for example, be constructed from a ceramic material or from a nitrided hardened stainless steel material.

The flexible conduit may, for example, be positioned within a sealed housing. A volume between the flexible conduit and housing may, for example, be filled with an aqueous fluid having dissolved solutes to provide an osmolarity approximately equal to the osmolarity of blood. The fluid may, for example, be an aqueous salt solution. The drive system may, for example, be in fluid connection with the volume, and the fluid may be present within the drive system. In a number of embodiments, the fluid is adapted to dissipate heat from the drive system. The fluid may, for example, include at least one hydrophilic lubricant.

In a number of embodiments, the flexible conduit is adapted to be placed in series with a blood vessel such as the aorta. The flexible conduit may, for example, be adapted to be placed in fluid connection with the ascending aorta. The flexible conduit may, for example, be adapted to be placed in line with a blood vessel such as the aorta (for example, the ascending aorta).

In another aspect, a method of assisting blood flow includes implanting a pump system within a body, wherein the pump system includes a flexible conduit, at least one moveable valve in fluid connection with the flexible conduit, a drive system including a rotary motor and a speed reducer operatively connected to the rotary motor, and a converter operatively connected to the drive system and to the valve to drive the valve in a reciprocating and linear manner or an approximately (or generally) linear manner.

In another aspect, a pump system for assisting blood flow includes a flexible conduit, at least one movable valve in fluid connection with the flexible conduit; and a sealed housing encompassing at least a portion of the flexible conduit. A volume between the housing and the flexible conduit includes an aqueous fluid having an osmolarity approximately equal to the osmolarity of blood. As described above, the fluid may, for example, be an aqueous salt solution. In a number of embodiments, the drive system is in fluid connection with the volume and the fluid is present within the drive system. The fluid may, for example, be adapted to dissipate heat from the drive system. The fluid may, for example, include at least one hydrophilic lubricant. The pump system may further include a drive system comprising a rotary motor and a speed reducer operatively connected to the rotary motor; and a converter operatively connected to the drive system and operatively connected to the valve to drive the valve in a reciprocating linear manner.

In another aspect, an implantable pump system for assisting blood flow includes a flexible conduit adapted to be placed in series with or in line with a blood vessel (for example, the aorta or the ascending aorta), at least one valve in fluid connection with the flexible conduit, a drive system including a rotary motor; and a converter operatively connected to the drive system and operatively connected to the valve to drive the valve in a reciprocating manner. The converter may be a rotary-to-linear converter adapted to drive the valve in a reciprocating, linear or approximately linear manner.

The drive system may further include a speed reducer operatively connected to the rotary motor, and the converter may be operatively connected to the speed reducer.

The pump system may further include a conduit housing encompassing at least a portion of the flexible conduit. A fluid may be present within the conduit housing outside of the fluid conduit. The fluid may, for example, be an aqueous salt solution. In a number of embodiments, the fluid has an osmolarity approximately equal to the osmolarity of blood.

The drive system may be in fluid connection with the conduit housing and the fluid may be present within the drive system. The fluid may be adapted to dissipate heat from the drive system. In a number of embodiments, the fluid includes at least one hydrophilic lubricant.

The pump system may further include at least one bearing fabricated from a material resistant to corrosion by a salt solution. The bearing may, for example, include a nitrided, martensitic steel or a ceramic material. In a number of embodiments, the bearing is a cageless roller element bearing. Toller elements of the cageless roller element bearing may, for example, include radiating gear teeth. The roller elements may further include at least one tapered, extending bearing surface.

In a number of embodiments, each mechanical linkage between the rotary motor and the valve comprises a cageless roller element bearing or a shielded roller element bearing. The cageless roller element bearing or the shielded roller element bearing may include at least one race formed of a nitrided, martensitic, stainless steel or a ceramic material and a plurality of roller elements formed from at least one of a nitrided, martensitic, stainless steel or a ceramic material.

In a number of embodiments, the speed reducing system includes a gear system. The speed reducing system may, for example, include a spur gear in operative connection with the rotary motor and a ring gear driven by the spur gear. The ring gear may be in operative connection with the rotary-to-linear drive system.

In a number of embodiments, the rotary-to-linear drive system includes an eccentric member extending from the ring gear. The rotary-to-linear drive system may further include a rotating element connected to the eccentric member that engages a cam member operatively connected to the valve to drive the valve in a reciprocating, linear manner.

In a number of embodiments, a rotor of the rotary motor extends generally perpendicular to a direction in which the valve is driven.

In a number of embodiments, the pump system includes a first housing section encompassing the flexible conduit and a second housing section encompassing the drive system. The second housing section extends from the first housing section at an angle thereto. The second housing section may extend from the first housing section at a right anterior oblique angle when the flexible conduit is placed in line with the ascending aorta.

In a number of embodiments, the valve of the pump system includes an opening and a plurality of closure members positioned within the opening that are rotatable to a closed position and to a range of open positions.

In another aspect, a method of assisting blood flow includes implanting a pump system within a body. The pump system includes a flexible conduit, at least one valve in fluid connection with the flexible conduit, a drive system including a rotary motor, and a converter operatively connected to the drive system and to the valve to drive the valve in a reciprocating manner. The flexible conduit of the pump system may, for example, be placed in series with or in line with a blood vessel (for example, the aorta or the ascending aorta).

The pump system may further include a first housing section encompassing the flexible conduit and a second housing section encompassing the drive system, wherein the second housing section extends from the first housing section at an angle thereto. The method may further include positioning the pump system so that the second housing section extends from the first housing section at a right anterior oblique angle when the flexible conduit is placed in line with the ascending aorta.

In a number of embodiments, a rotor of the rotary motor extends generally perpendicular to a direction in which the valve is driven. In a number of embodiments, the converter is a rotary-to-linear converter and drives the valve in a reciprocating, linear manner. The rotor may, for example, extend in a right anterior oblique direction into a space normally occupied by the right lung.

The method may further include filing a free volume within the first housing section outside of the flexible conduit and a free volume within the second housing section with a fluid. The free volume of the first housing section and the free volume of the second housing section may be in fluid connection. In a number of embodiments, the fluid is an aqueous salt solution. The fluid may, for example, have an osmolarity approximately equal to the osmolarity of blood. The fluid may, for example, be adapted to dissipate heat from the drive system.

In another aspect, a pump system for assisting blood flow, includes a flexible conduit, at least one valve in fluid connection with the flexible conduit, a conduit housing encompassing at least a portion of the flexible conduit, a drive system operatively connected to the valve to drive the valve, a drive system housing encompassing at least a portion of the drive system in fluid connection with the conduit housing; and an aqueous fluid within the conduit housing outside of the flexible conduit and within the drive system housing. The aqueous fluid may, for example, be a solution comprising at least one salt. The aqueous fluid may, for example, have an osmolarity approximately equal to the osmolarity of blood. The fluid may also be adapted to dissipate heat from the drive system. In a number of embodiments, the fluid includes at least one hydrophilic lubricant. The pump system may further include at least one bearing fabricated from a material resistant to corrosion by a salt solution.

In a number of embodiments, the drive system includes a rotary motor, a speed reducing system in operative connection with the rotary motor and a convertor operatively connected to the speed reducing system. The converter is operatively connected to the valve to drive the valve in a reciprocating manner. The converter may, for example, be a rotary-to-linear converter which drives the valve in a reciprocating, linear manner

In a further aspect, a pump system for assisting blood flow, includes a flexible conduit adapted to be placed in series with or in line with a blood vessel such as the aorta (for example, the ascending aorta), at least one movable valve in fluid connection with the flexible conduit, and a sealed housing encompassing at least a portion of the flexible conduit. A volume between the housing and the flexible conduit includes an aqueous fluid having an osmolarity approximately equal to the osmolarity of blood.

In still a further aspect, a pump system for assisting blood flow, includes a flexible conduit, at least one valve in fluid connection with the flexible conduit, a drive system; and a converter operatively connected to the drive system and operatively connected to the valve to drive the valve in a reciprocating manner. Moving mechanical linkages of the drive system and between the drive system and the valve each include at least one rolling element bearing. At least one of the rolling element bearings includes races having gear teeth and a plurality of roller elements including cooperating gear teeth to maintain relative positions of the roller element bearings. In a number of embodiments, the roller elements are formed from at least one of a martensitic, nitrided, hardened stainless steel or a ceramic material.

In a number of embodiments, the projected average life of pump systems hereof is intended to be that of current heart transplants, namely approximately 10 years.

The technology described herein, along with the attributes and attendant advantages thereof, will best be appreciated and understood in view of the following detailed description taken in conjunction with the accompanying drawings.

### BRIEF DESCRIPTION OF THE DRAWINGS

FIG. 1 illustrates a perspective view of the human heart including some of the surrounding vasculature and some of the surrounding organ structure.

FIG. 2 illustrates a perspective view of an embodiment of a pump system hereof placed in line with the ascending aorta.

FIG. 3A illustrates a front view of the pump system of FIG. 2 with closure members of the valve assembly of the pump system in a partially open state.

FIG. 3B illustrates a perspective view of the pump system of FIG. 2.

FIG. 3C illustrates a side view of the pump system of FIG. 2.

FIG. 4A illustrates another side view of the pump system of FIG. 2.

FIG. 4B illustrates a cross-sectional view of the pump system of FIG. 2 along section A-A of FIG. 4A wherein the valve closure members are in a closed state.

FIG. 4C illustrates a cross-sectional view of the pump system of FIG. 2 along section A-A of FIG. 4A wherein the valve closure members are in an open state.

FIG. 4D illustrates an embodiment of a drive system for use in pump systems hereof.

FIG. 4E illustrates a side view of the drive system of FIG. 4D.

FIG. 4F illustrates a cross sectional view of the drive system of FIG. 4D along section C-C.

FIG. 4G illustrates several geared roller elements suitable for use in rolling element bearing of the drive system of FIG. 4D in various orientations.

FIG. 4H illustrates a front view of the drive system of FIG. 4D.

FIG. 5A illustrates a side view of an embodiment of a shaft for a rotary motor for used in pump system hereof in operative connection with a rolling element bearing.

FIG. 5B illustrates a rear view of the shaft and bearing assembly of FIG. 5A.

FIG. 5C illustrates a cross-sectional view of the shaft and bearing assembly of FIG. 5A along section D-D of FIG. 5A.

FIG. 5D illustrates a rear perspective view of the shaft and bearing assembly of FIG. 5A.

FIG. 5E illustrates a perspective cutaway view of the shaft and bearing assembly of FIG. 5A wherein the assembly is cut along section D-D of FIG. 5A.

FIG. 5F illustrates a perspective, exploded or disassembled view of the shaft and bearing assembly of FIG. 5A.

FIG. 6A illustrates a perspective, exploded or disassembled view of the pump system of FIG. 2 with the housing sections removed.

FIG. 6B illustrates a perspective, exploded or disassembled view of a flow conduit assembly and curved connector of the pump system of FIG. 2 with the housing section of the flow conduit removed.

FIG. 6C illustrates a perspective view of the flow conduit assembly and curved connector of the pump system of FIG. 2 (with the housing section of the flow conduit removed) and an embodiment of a converter for converting rotary motion to reciprocating motion.

FIG. 6D illustrates a perspective view of the flow conduit assembly and curved connector of the pump system of FIG. 2 (with the housing section of the flow conduit removed) and another embodiment of a converter for converting rotary motion to reciprocating motion.

FIG. 6E illustrates a perspective view of an embodiment of a race member and roller elements thereof for use in a linear bearing.

FIG. 7A illustrates a perspective view of an embodiment of a linear rolling element bearing for use in the pump systems hereof.

FIG. 7B illustrates a top view of the linear rolling element bearing of FIG. 7A.

FIG. 7C illustrates a side view of the linear rolling element bearing of FIG. 7A.

FIG. 7D illustrates a front view of the linear rolling element bearing of FIG. 7A.

FIG. 7E illustrates several geared roller elements suitable for use in the linear rolling element bearing of FIG. 7A.

### DETAILED DESCRIPTION

As used herein and in the appended claims, the singular forms “a,” “an”, and “the” include plural references unless the content clearly dictates otherwise. Thus, for example, reference to “a valve assembly” includes a plurality of such valve assemblies and equivalents thereof known to those skilled in the art, and so forth, and reference to “the valve assembly” is a reference to one or more such valve assemblies and equivalents thereof known to those skilled in the art, and so forth.

In a number of embodiments hereof devices, systems and methods are disclosed for assisting blood flow using a moving (for example, oscillating) valve assembly or a plurality of such valve assemblies to propel blood. Pump systems hereof may be fully implanted or temporarily connected to the circulation using percutaneous blood conduits. Pump systems hereof may, for example, be fully or completely implanted for a period of months to years to alleviate or correct heart failure and related symptoms.

Inline moving valve pumps located in the space normally occupied by, for example, the ascending human aorta have been proposed in which a linear motor actuates valve movement. As used herein, the terms “in line or “inline” when used in connection with the position of a pump system hereof within the body refers to a moving valve pump in which the flow path of blood through the pump is in series with a blood vessel (for example, the aorta or the ascending aorta) and the path of the moving valve passes through at least a portion of the volume occupied by the blood vessel (for example, the aorta or the ascending aorta) prior to implantation of the moving valve pump. Because of the critical organs surrounding the ascending human aorta, there is very limited space for a valve drive mechanism. A compact linear motor was previously believed to be required for use in the limited space around the aorta. Moreover, the ascending aorta is typically three inches or less in length (approximately two inches in length for an average adult), which is a very short distance for the provision of a flow conduit assembly of an inline moving valve pump system (for example, including one or more flexible conduits, sealing end rings, and blood tight aortic connections at each end of the pump system). The present inventors have discovered that suitably sized linear motors have inadequate output power for driving a moving valve pump at motor temperatures reasonably suitable for implantation in the human body, and, for example, to be in series with and/or in line with a blood vessel such as the aorta (for example, the ascending aorta).

Rotary motors have the advantage of higher power compared to an equally sized linear motor. However, implantable rotary motors for an inline moving valve pump would have to be placed in the vicinity of the aorta, consuming organ space. In a number of embodiments hereof, pump systems having a drive system including a rotary motor and a converter (to convert the rotary motion of the rotary motor to the desired reciprocating motion of the moving valve) exhibit sufficient driving power for the moving valve with acceptable heat emission. A rotary-to-linear drive or converter can be used for reciprocating, linear valve motion, or a rotary-to-curvilinear drive or convertor can be used for reciprocating, curvilinear valve motion. A speed reduction system or speed reducer can, for example, be provided between the rotary motor and the converter. Such a speed reduction system can, for example, be used to decrease the size (volume) of the rotary motor required. Representative embodiments of pump systems hereof exhibit a form factor that does not significantly interfere with the function of the critical organs surrounding, for example, the aorta.

Review of the physics describing motor power illustrates the power output advantages of a rotary motor compared to a linear motor, and particularly, a rotary motor including a speed reducer. Output power is the product of torque and rotational speed for a rotary motor, while power is the product of force and velocity in the case of a linear motor. Electromagnetically induced force, expressed in Newtons, is the product of magnetic field strength expressed in Tesla, the length of an electric conductor in meters, and the current flowing in the conductor expressed in amperes, provided the current is flowing perpendicular to the magnetic flux lines. If one assumes an equal volume of copper for carrying current and a rare earth magnet material such as neodymium, for establishing magnetic field strength, equally sized linear and rotary motors will be capable of creating roughly the same amount of electromagnetic force. However, the generated power will be the product of this force and the corresponding velocities. A linear motor, directly linked with a valve might, for example, move the valve 0.015 meters (1.5 centimeters) in 50 milliseconds for a velocity of approximately 0.3 meters per second. A rotary motor linked to the valve through, for example, a 5×speed reducer, and a rotary-to-linear converter (for example, including a cam element), and having a rotor diameter of 0.02 meters (2 centimeters), will have rotor surface motion per valve stroke of (pi radians×0.01 meters/radian×5)/0.05 or 3.14 meters per second. In this representative example, the rotary motor thus provides more than ten times the velocity of the linear motor. Therefore, for an equivalent mass and size of linear and rotary motors, the rotary motor can be expected to produce roughly ten times the output power compared with the same input power. The substantial difference in power generation efficiency allows use of a suitably small rotary motor, especially when coupled with a speed reducer to perform the required work of a moving valve pump located in the limited space of the ascending aorta, with acceptable heat generation. A linear motor suitable to perform the same amount of work would have to be too large and/or would emit too much heat.

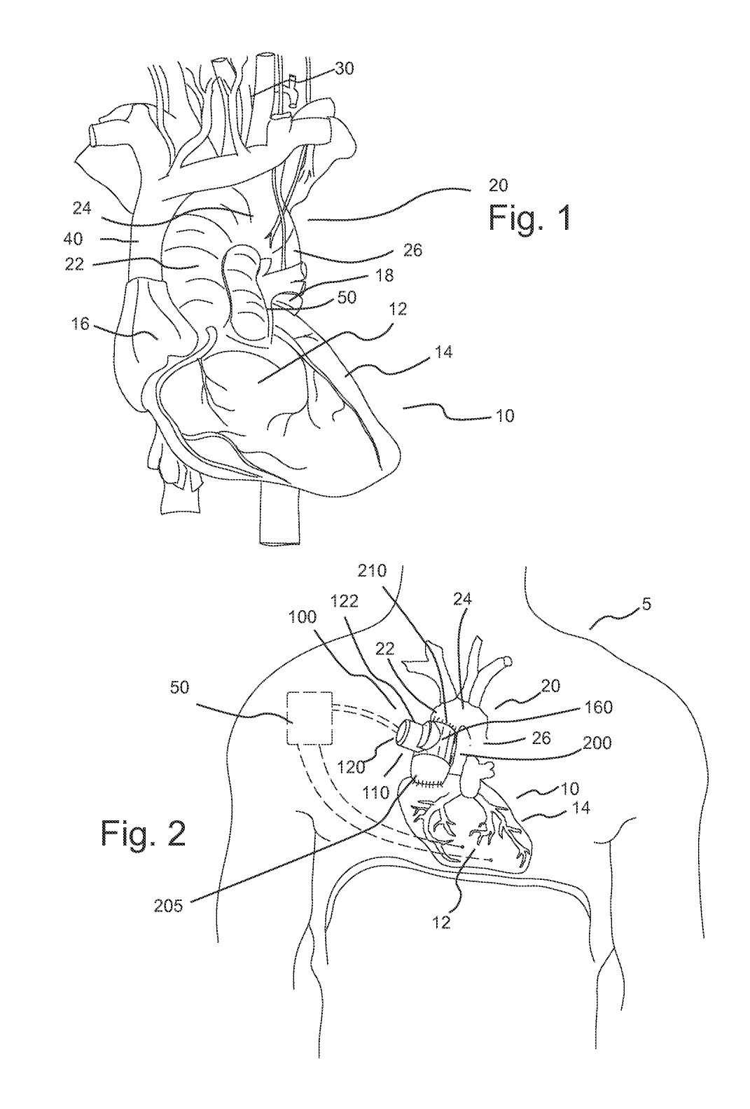

FIG. 1 illustrates a frontal view of human heart 10, including some of the surrounding vasculature and other surrounding organ structures. FIG. 1 does not show the bony thorax, including the sternum and attached ribs, that limit the space immediately in front of heart 10 and associated structures. Heart 10 includes right ventricle 12 and left ventricle 14. As described above, left ventricle 14 is the main pumping chamber of heart 10. During left ventricular contraction or systole, blood is pushed through the aortic valve into ascending aorta 22, which is the main artery leading to the body. In the vicinity of heart 10, aorta 20 includes the ascending aorta 22, the arch of aorta or aortic arch 24 and the descending aorta 26. For placement of a pump system in line with ascending aorta 22, critical peri-aortic structures include, but are not limited to, right atrium 16 and left atrium 18, esophagus 30, superior vena cava 40, the pulmonary veins (not shown) and pulmonary artery 50. Left ventricle 14 normally has a conical form, the long axis of which is generally in line with the root of ascending aorta 22 Pulmonary artery 50 and the right branch thereof, wrap around and behind ascending aorta 22. Right atrium 16, or entrance chamber to right ventricle 12, bounds the lower portion of ascending aorta 22 on its right side (with reference to the orientation of patient 5). In the illustration of FIG. 1, superior vena cava 40 bounds the higher portion of ascending aorta 22 to its right (with reference to orientation of patient 5).

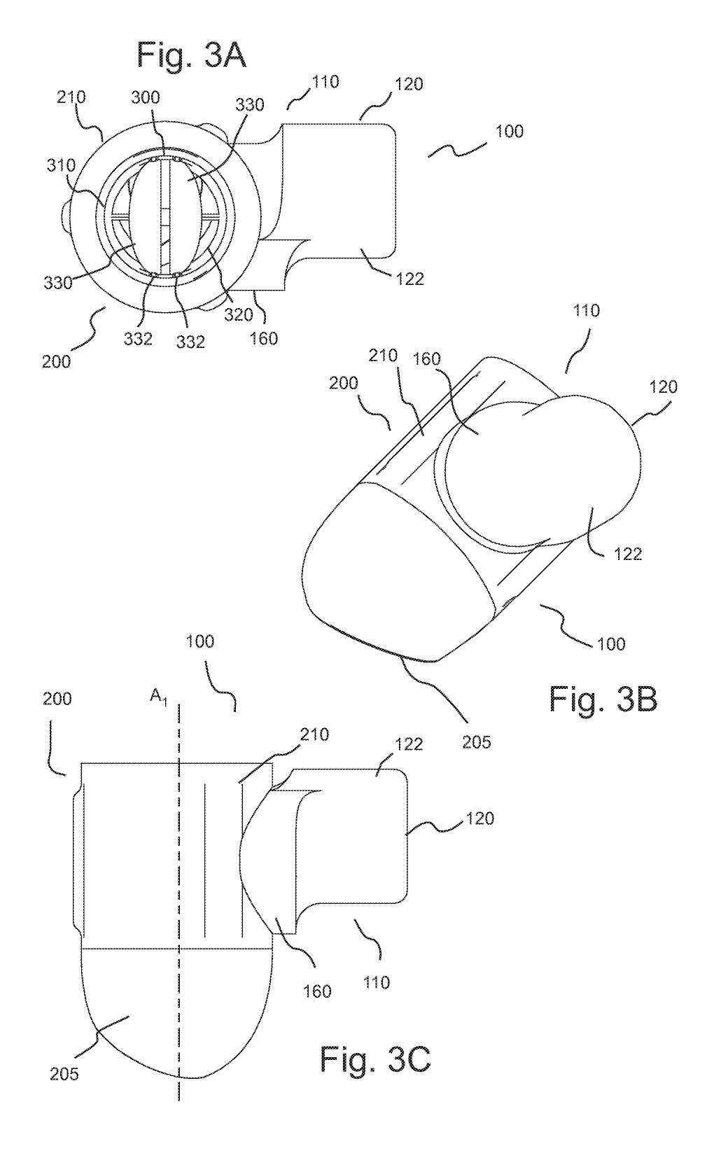

FIG. 2 illustrates an embodiment of an inline pump system 100 hereof wherein a blood flow path of pump system 100 is placed in line with ascending aorta 22. Some of the vasculature and structures surrounding the heart illustrated in FIG. 1 are removed in FIG. 2. Because of the bony thorax, limiting the space in front of heart 10 as illustrated in FIGS. 1 and 2, drive system 110 of pump system 100, including rotary motor 120 and connected speed reducer 160 in the illustrated embodiment (see, for example, FIGS. 3A through 3C), protrude sideways at a right anterior oblique angle (with reference to the orientation of patient 5) from conduit assembly 200 through which blood flows, which is a generally cylindrical structure over a portion thereof. In the frontal view of FIG. 2, rotary motor 120 protrudes to the left (of the viewer of FIG. 2, opposite the reference with respect the orientation of patient 5) of the upper end of ascending aorta 22 to avoid interfering with the bony thorax and vital surrounding structures. With respect to critical peri-aortic structures, there is suitable space for pump system 100 at the upper end of ascending aorta 22 and toward the right lung (not shown), where the right lung is in close proximity to ascending aorta 22. This right lung space can be occupied by the protruding portion of drive system 110 without significant health consequences or significant spatial interference with other vital structures. In the illustrated orientation, drive system 110 can be sufficiently small in volume to displace only a small amount of volume of the right lung and sufficiently short in axial length to not interfere with the inner surface of the bony sternum that bounds this peri-aortic space anteriorly.

FIG. 2 illustrates schematically a control system 50 in operative connection with pump system 100. Control system 50 can, for example, be implanted subcutaneously at a position remote from pump system 100 in the upper chest of the patient and placed in communicative connection with pump system 100 (for example, via wiring). Control system 100 includes control algorithms for valve movement of pump system 100, and can, for example, include a microprocessor-based position servo control system. Heart rhythm can, for example, be used to time the valve oscillations. In FIG. 2, leads to heart 10 provide a signal of the heart's rhythm to control system 50.

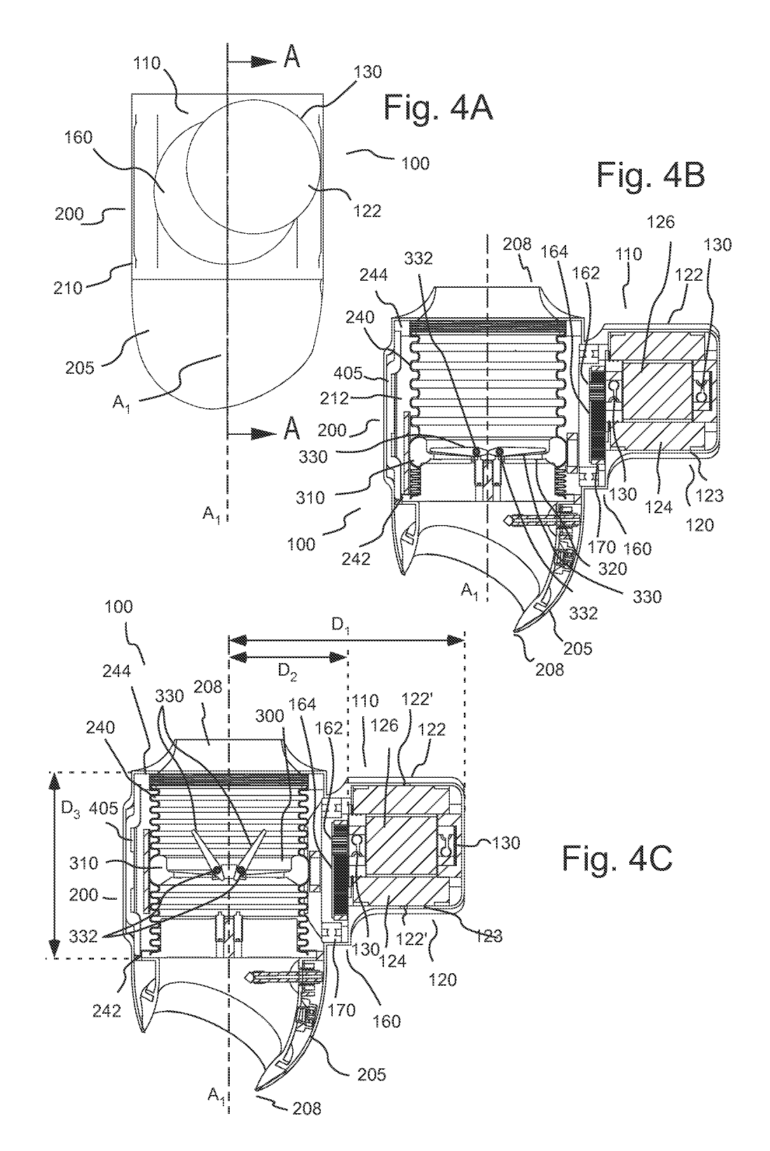

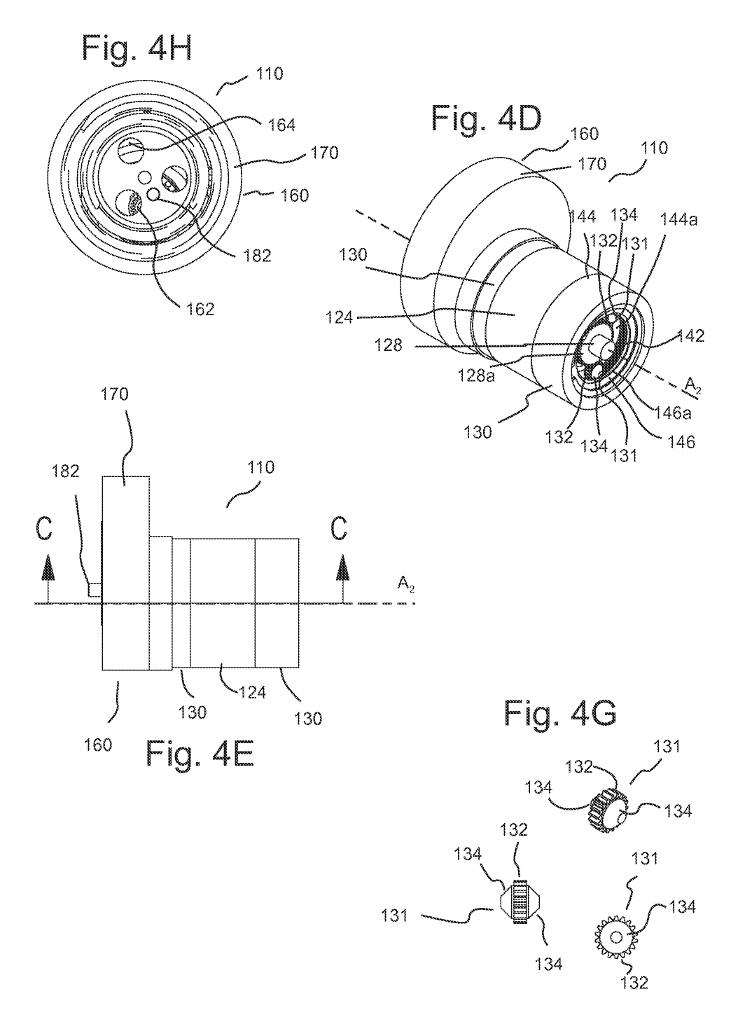

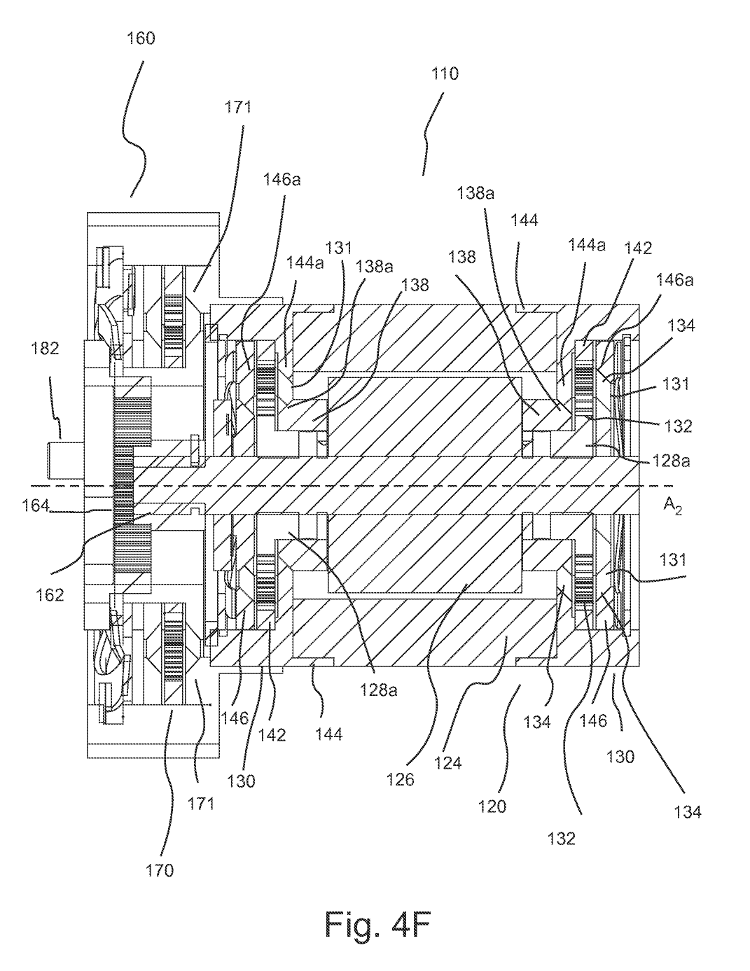

FIGS. 3A through 4C illustrate moving valve pump system 100 in various orientations. As described above, flow conduit assembly 200 can be generally cylindrical in shape. Rotary motor 120 and speed reducer 160 can also be generally cylindrical in shape. In a number of embodiments of pump systems hereof, a housing or housing section of the flow conduit assembly is adjacent to and extends at an angle from a housing or housing section of the drive system. The housing sections can be formed separately and connected or formed as an integral or monolithic housing or case. In a number of embodiments, the axis of the rotary motor (see axis A₂ in FIGS. 4D and 4F) within the housing section for the drive system extends to intersect the housing of the flow conduit assembly. In the embodiment of FIGS. 3A through 4C, a housing section 122 of drive system 110 extends generally perpendicular to a housing section 210 of flow conduit assembly 200. Axis A₂ of shaft 128 of rotary motor 120 extends generally perpendicular to axis A₁ of flow conduit assembly 200 (which is also the axis or centerline of blood flow through flow conduit assembly 200 in the illustrated embodiment). The conformations or form factors described above assist in reducing or minimizing the volume of the pump system and in reducing, minimizing or eliminating interference with vascular structure and organ structure in the vicinity of aorta 20.

The total volume displaced by the pump system can, for example, be less than 400 cc, less than 200 cc or even less than 180 cc. The volume of flow conduit assembly 200 and the extending housing section of the drive system may, for example, be less than 300 cc, less than 200 cc or even less than 120 cc. The volume displaced by the extending housing section of the drive system may, for example, be less than 150 cc, less than 100 cc or even less than 50 cc. A distance D₁ (see FIG. 4C) from axis A₁ (or the centerline of blood flow) to a distal end of the housing section for the drive system (housing section 122 in the embodiment illustrated in FIGS. 3A through 4C) may, for example, be less than 12 cm, less than 10 cm or even less than 8 cm. A distance D₂ (see FIG. 4C) from axis A₁ (or the centerline of blood flow) to the position at which the motor shaft exits the body of the motor (typically, at the face of a bearing on an axial end of the motor) may, for example, be less than 6 cm or less than 4 cm. A distance or length D₃ (see FIG. 4C) corresponding to the length of flow conduit assembly 200 (through which a valve assembly 300 moves in a reciprocating manner) extends can, for example, be less than 7.6 cm, less than 6.4 cm or even less than 5 cm.

In one embodiment, the total volume of pump system 100 is approximately 170 cc, the volume of flow conduit assembly housing section 210, a curved connector 205 attached thereto and a suture connector 208 attached thereto is approximately 136 cc; the volume of flow conduit assembly housing section 210 is approximately 79.08 cc; the volume of drive system housing section 122 (encompassing rotary motor 120 and speed reducer 160) is approximately 34 cc; distance D₁ is approximately 7.75 cm; and distance D₃ is approximately 4.52 cm. In that embodiment, the volume of aorta 20 displaced by pump system 100 is approximately 75 cc (calculated using an average aorta radius of 1.75 cm). The net increase in volume which impinges on non-aortic structures is approximately 95cc (170 cc-75 cc).

As described above, in the embodiment illustrated, for example, in FIGS. 3A through 4C, drive system 110 (including rotary motor 110 and speed reducer 160 and housing section 122 therefor) is attached generally perpendicular to an axis A₁ (see, for example, FIGS. 4A and 4C) of a housing section 210 of conduit assembly 200 through which a valve assembly 300 moves in the direction of axis A₁ to assist blood flow. Axis A₂ of shaft 128 (see, for example, FIG. 4F) of rotary motor 120 and the axis of ring gear 164 of speed reducer 160 are generally perpendicular to axis A₁ of flow conduit assembly 200. As also described above, upon implantation, drive system 110 can be oriented in the right anterior oblique direction into the space normally occupied by the right lung. If drive system 200 was oriented on the anteroposterior axis (that is, aimed straight out of the illustration toward the viewer in FIG. 2), it would interfere with the bony thorax covering heart 10 and ascending aorta 22.

FIGS. 4B and 4C illustrate cross-sectional views of moving valve pump system 100. Rotary motor 120 can, for example, be a brushless direct current motor positioned within housing section 122 (which can be generally cylindrical over at least a portion thereof). A stator 124 of rotary motor 120, as its name implies, is stationary and physically connected to mechanical ground. As used herein, the term “mechanical ground” refers to a non-moving portion of a system or subsystem being discussed. As known in the motor arts, stator 124 can, for example, include a series of electromagnets arranged in a continuous circle that are electronically activated in a carefully timed sequence to rotate a rotor 126. Rotor 126 can, for example, include rare earth magnets located on its periphery that interact with electromagnets of stator 124 to produce the torque force necessary to rotate rotor 126. As described further below, shaft 128 (see, for example, FIG. 4F) of rotor 126, and consequently rotor 126, can, for example, be suspended or positioned within motor housing 122 by rolling element bearings 130.

In the illustrated embodiment, speed reducer or speed reduction system 160 is in operative connection with rotor shaft 128. In FIGS. 4A through 4C, housing 122 houses both the components of rotary motor 120 and speed reducer 140. As described above, housing section 122 can be attached to housing section 210 or formed at least partially integrally or monolithically therewith to form a housing or case for pump system 100.

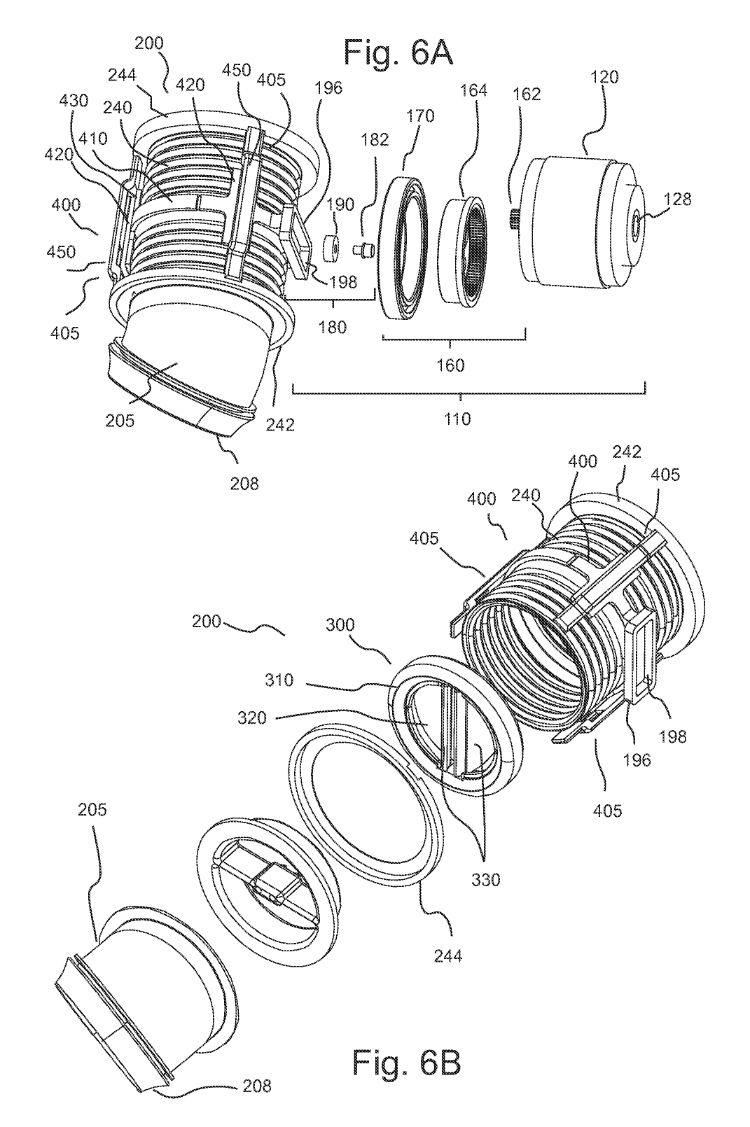

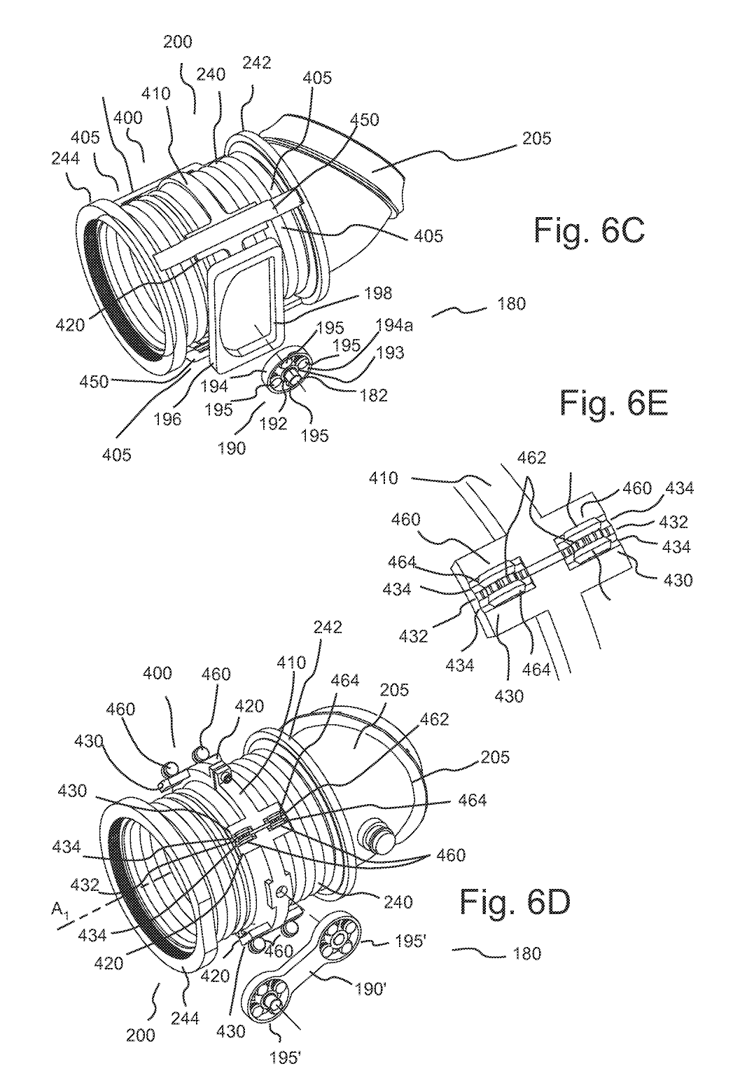

In the illustrated embodiment, speed reducer 160 includes a spur gear or a pinion gear 162 attached to rotor shaft 128. Spur gear 162 engages and rotates a ring gear 164. Ring gear is suspended or positioned by a bearing such as a rolling element bearing 170 including roller elements 171 as described below in connection with rolling element bearings 130 of rotary motor 120. The combination of spur gear 162 and ring gear 164, provide a number of advantages over, for example, a planetary speed reducer because of the need for bushings or bearings for each planet gear of the planetary speed reducer and the additional need for a power takeoff from the centers of each of the planets thereof. Using spur gear 162 to drive ring gear 164 is much simpler, and requires only two gears for achieving a suitable speed reduction. In the illustrated embodiment, speed reducer 160 is operatively connected to a rotary-to-linear converter 180 (see, for example, FIGS. 6A through 6E). Rotary-to-linear converter 180 is operatively connected to valve assembly 300 to drive valve assembly 300 in a linear reciprocating manner by converting the rotating motion of ring gear 164 to linear, reciprocating motion.

As described above, speed reducer 160 can be eliminated in the case of certain rotary motors. In such an embodiment, the rotary motor would be connected directly to the converter to convert rotary motion to the reciprocating motion of the valve assembly. However, a rotary motor providing sufficient torque at lower speeds would be required. Such a rotary motor would have a substantially increased volume and weight as compared to a rotary motor suitable for use in connection with a speed reducer.

The magnitude of the lifetime requirements for a moving valve pump which is intended to be an alternative to a heart transplant is substantial. Although, heart transplants are very effective solutions for severe heart failure, heart transplants are limited by availability of suitable donor hearts (approximately 2,400 per year in the US). The need for a heart transplant equivalent replacement pump is therefore great. However, the desired lifetime for a pump system equivalent to a heart transplant is on the order of ten years of very reliable operation. If a moving valve pump system such as pump system 100 averages three cycles/forward strokes per heartbeat, and the average heart rate of a pump recipient patient is 80 beats per minute, 1.26 billion reliable valve cycles are required from the pump system 100 (10 years×365 days/year×24 hours/day×60 minutes/hour×80 heartbeats/minute×3 cycles/heartbeat). This is a high number of cycles for reliable operation of mechanical linkages from rotary motor 120 to valve assembly 300.

Transferring energy from rotary motor 120 to valve assembly 300 with such a long lifetime requirement presents a problem of wear of bearings used in pump system 100. As used herein, a “bearing” refers to a device that allows constrained relative motion between two or more components (most commonly, rotational or linear movement). Bushings, which are independent plain bearings inserted into a housing to provide a bearing surface for rotary or linear applications, can be overly susceptible to wear. In general, sliding or friction bearings are subject to wear and would be expected to decrease pump system lifetime. Rolling element bearings carry a load by placing rolling elements between two race components. The relative motion of the components causes the rolling elements to roll with little resistance. Rolling element bearing can provides improved wear resistance as compared to bushings. A ball bearing is a type of rolling element bearing in which balls maintain separation between the moving parts of the bearing. Because of cost and availability reasons, ball-shaped rolling elements are typically used in motors to link mechanical assemblies. However, ball-shaped rolling elements provide a single point of contact with the moving component(s) of the bearing. Rolling element bearings, which provide a generally linear contact with moving parts (for example, cylindrical rolling elements), exhibit higher load carrying capability and can provide improved wear resistance compared to some ball bearings. A problem with a number of currently available rolling element bearings, given the relatively long lifetime requirements for pump system 100, is the use of cages to maintain proper placement of the rolling elements. Because rubbing between the cage and the rolling elements is unavoidable and generates wear and debris, rolling element bearings which do not include cages (sometimes referred to herein as cageless rolling element bearings) are used in several embodiments hereof Cageless rolling element bearings can, for example, be provided by using matched or meshing gear teeth on the rolling elements and the cooperating races to properly locate the rolling elements within the rolling element bearing assemblies. Since the respective gear teeth of the rolling elements and races mesh, rubbing is virtually eliminated and bearing life is increased. Additionally, roller element bearings with extending bearing surfaces can be used instead of balls for the rolling elements in at least some of the mechanical linkages of pump systems hereof. As described above, rollers provide greater load bearing capacity compared to balls, which provide only point contact for load bearing. Rolling elements or rollers with extended contact bearing surfaces for use herein can, for example, include angled, tapered, canted or arced extending bearing surfaces to better accommodate bending moments and stresses on the rolling element bearings.

Certain caged rolling element bearings, including ball bearings, may also be suitable for use in at least some of the mechanical linkages of the pump systems hereof. For example, “hybrid” rolling element bearings are available wherein the inner and outer races are formed from a bearing hard steel, while the rolling balls are formed from a ceramic material such as silicon nitride. Hard ceramic balls, formed from silicon nitride are lighter than steel and have a higher modulus of elasticity, which makes them stiffer than steel. Moreover, ceramic balls are smoother than steel balls and do not microscopically weld to the steel races because of the dissimilarities of the materials. Hybrid rolling element bearings including ceramic rolling balls or rollers and durable cages are, for example, available from The Barden Corporation of Danbury, Conn. Durable or long-life cages can, for example, be formed from a thermoplastic material.

Whether the bearings used in the pump systems hereof are caged or cageless, and whether such bearings include balls or rollers with extending bearing surfaces, such bearing can, for example, include shielding. Shielding (for example, a metallic cover or shield) covers the rolling elements and races of shielded bearings. Typically, shielding is used to keep debris from entering the bearing or to retain a lubricant such as grease within the bearing. In the case of bearings used in the pump systems hereof, however, shieling can be used to prevent debris caused by wear of one or more components of the bearing from escaping the bearing. Typically, shielding on both sides of a bearing is desirable.

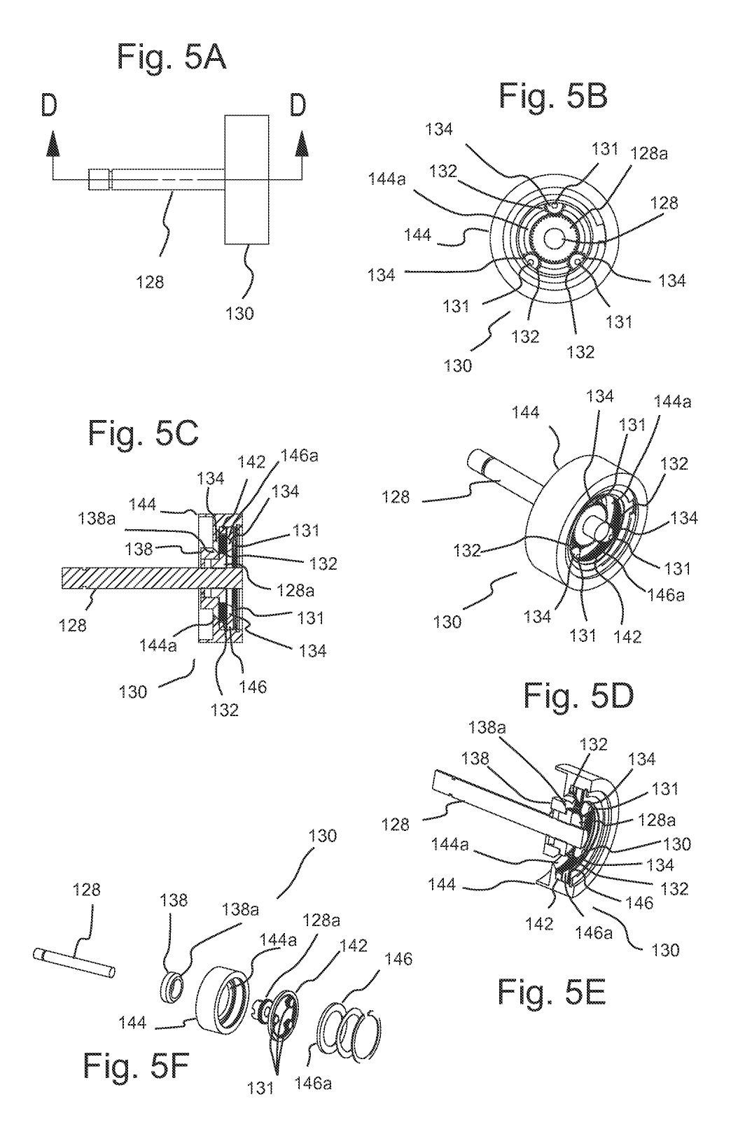

FIGS. 4D through 4G illustrate drive system 110 wherein rotary motor 120 is slightly different in appearance from rotary motor 120 illustrated in FIGS. 4A through 4C, but drive systems 110 operates in the same manner as described above. The appearance of rotary motor 120 in FIGS. 4A through 4G can, for example, provide a better view of the operation of one embodiment of rolling element bearings used therein (see, for example, FIGS. 4D and 5A through 5F).

As described above, rotor shaft 128 is supported by rolling element bearings 130. In the illustrated embodiment of, for example, in FIG. 4G. rolling elements 131 of rolling element bearings 130 include a gear or toothed section 132 including radiating gear teeth. Rolling elements 131 further include angled or tapered bearing surfaces 134 extending on each lateral side of gear section 132.

Rotary shaft 128 has two relatively larger gear wheels 128a keyed or otherwise fixed thereto. The geared teeth of roller elements 131 cooperate with the teeth of gear wheels 128a. FIGS. 5A through 5F illustrates several views of shaft 128 of rotary motor 120 with one rolling element bearing 130 in operative connection therewith. To form a radially inward race of bearing 130, a bearing member 138 (see, for example, FIGS. 5C, 5E and 5F) having an angled or tapered bearing surface 138a can be positioned on one or both sides of gear wheel 128a to provide radially inward surfaces for angled bearing surfaces 134 of roller elements 131 to bear against. Rotary motor 120 further includes a radially outward positioned race to cooperate with roller elements 131. As illustrated, for example, in FIGS. 4D and 5B through 5F, the radially outward positioned race is formed by an intermediate gear ring 142 including radially inward radiating gear teeth and end rings 144 and 146 which include angled or tapered bearing surfaces 144a and 146a, respectively. End ring 144 also provides a seating for gear ring 142 and end ring 146.

As illustrated, for example, in FIGS. 4D, and 5B through 5F) rolling elements 131 are arranged around gear wheel 128a. Bearing surfaces 138a. 144a and 146a are adapted to support shaft 128 and the elements attached thereto. The intermeshing teeth of gear wheel 128a, roller elements 131 and gear ring 142 operate to prevent relative movement of roller elements 131 about the inner and outer races of rolling element bearing 130, thereby obviating the need for cages in the embodiment of bearing 130.

As illustrated, for example, in FIGS. 4B, 4C, and 6A through 6D, a blood flow path of pump system 100 which includes at least one flexible blood flow conduit 240 (which can, for example, be corrugated or otherwise folded to provide flexibility over at least a portion thereof) can, for example, be placed in line with ascending aorta 122. Blood flows through flow conduit 240 and is driven by moving valve assembly 300 (see, for example, FIGS. 4B, 4C and 6B). Flow conduit 240 is designed and constructed of a flexible material so that it can be extended and compressed along its length as valve assembly 300 moves forward and rearward (for example, in an oscillating manner). Flow conduit 240 can, for example, operate in the manner of a bellows and is sometimes referred to as such herein. Valve assembly 300 can, for example, be attached (for example, by an adhesive) via a peripheral support structure or valve ring 310 thereof to a single flow conduit 240. Alternatively, one flow conduit 240 can be attached to a rearward end of valve support structure 310 and another flow conduit 240 can be attached to a forward end of valve support structure 310. As described above, the walls of the flow conduit 240 can be corrugated, stretchable or otherwise moveable along the path of movement of valve assembly 300 to allow movement (for example, via contraction and expansion) of flow conduit 240 as valve assembly 300 moves in a reciprocating or back-and-forth linear manner. In a number of embodiments, flow conduit 240 is formed from a flexible, durable, bio/blood compatible material such as a metal or a polymeric material which exhibits in vivo biostability over the life of pump system 100. In a number of embodiments wherein flow conduit 240 is formed of a polymeric material, the polymer is a urethane polymer, which can be elastomeric. The conduit wall material of flow conduit 240 can, for example, be BIONATE® urethane polymer, which is a durable implant grade, bio/blood compatible thermoplastic polycarbonate urethane available from Polymer Technology Group, Inc. of Emeryville, Calif. In a number of embodiments, a generally consistent wall thickness is maintained over the length of flow conduit 240 to avoid weak areas in flow conduit 240. For example, in a number of embodiments, flow conduit 240 was formed from BIONATE to have a wall thickness of 10 mils±2 mils (0.254 millimeters±0.051 millimeters).

Flow conduit assembly 200 can, for example, include sealing end rings 242 and 244 to which the ends of flow conduit 240 are attached. Rearward (relative to the flow of blood through flow conduit 240 from heart 10) end ring 244 can, for example, have attached thereto a curved connective portion 205 for connection to ascending aorta 22 which corresponds generally to the form of ascending aorta 22 as it is connected to heart 10 and assists in positioning the blood flow path of conduit assembly 200 in line with ascending aorta 22. A connector such as suturable connectors 208 (illustrated in FIG. 4B, which can, for example, be formed of a polymeric material such as a urethane polymer) for connecting end ring 244/flow conduit 240 to ascending aorta 22 can, for example, be placed in sealed connection with end ring 244.

A space or volume 212 surrounding flow conduit 240 and bounded and sealed by housing 210 can, for example, be filled with a fluid (that is, a liquid, sometimes referred to herein as the peri-bellows fluid). The fluid can, for example, operate, in part, to equalize pressure within housing 210 and outside of flow conduit 240 with the pressure within flow conduit 240. The fluid in space 212 can, for example, be a blood compatible, aqueous salt solution (including, for example, sodium chloride). In a number of embodiments, the salt solution has an osmotic pressure, an osmolarity or an osmolality approximately equal to that of blood (for example, an osmolarity within 5% or even within 2% of the osmolarity of blood).

As described above, in the case that, for example, a semipermeable material (such as an elastomeric polymer) is used for fluid conduit 240, the fluid/liquid may, for example, exhibit a similar osmolarity/osmotic pressure to that of blood to, for example, prevent fluid from moving into or out of peri-bellows space 212. Such fluid movement could, for example, damage flexible fluid conduit or bellows 240 either by the fluid conduit 240 rubbing against case or housing 210 upon fluid volume shrinkage or crinkling of fluid conduit 240 upon fluid volume expansion. Fluid movement into or out of the housing of pump system 100 (and thus a change of the volume of fluid within the housing) can occur because of the semi-permeable nature of polyurethane and/or other materials (in which a fluid can be transported through the material) which may be used in flexible fluid conduit 240 where there is a significant osmotic pressure difference across flexible conduit 240. Human blood contains 292 plus or minus ˜12 milliosmols of osmotic pressure. A sodium chloride aqueous solution of approximately 0.852% by weight approximately matches blood osmotic pressure and can be used to limit or prevent fluid movement into or out of the space 212. Further, aqueous salt solutions such a sodium chloride aqueous fluid solution in space 212 also provides the advantage of being biocompatible if released into the blood stream.

In the case that fluid conduit 240 is impermeable to the aqueous fluid/liquid surrounding flow conduit 240, it may not be necessary to match the osmolarity of the aqueous fluid to that of blood. In the case that a semipermeable polymeric material such as BIONATE is used for fluid conduit 240, the outer surface thereof can, for example, be surrounded with or coated by a layer of a flexible, impermeable polymer or other impermeable material to reduce or eliminate permeability.

In a number of embodiments, the fluid within space 212 also surrounds the components of rotary-to-linear converter 180 and the components of speed reducer 140 and also fills the space of volume between stator rotor 124 and rotor 126. The fluid can also fill a space 123 between the rotary motor components (for example, bearings 130 and stator 124) and housing 123. Rotary motor 120 can, for example, be spaced from housing 122 and fixed or stabilized in position relative thereto by spacer or standoffs 122′ (see FIG. 4C) which can, for example, be formed from an insulating material. Gases can, for example, be eliminated from all volumes within pump system 100, including from within housing 210 and housing 122. As known to those skilled in the medical arts, release of a gas into the bloodstream can have serious adverse consequences. Oily fluid or hydrophobic lubricant, normally used within motors and/or mechanical linkages can also be injurious because oil/hydrophobic lubricant, upon release, can flow downstream in the arterial system and can cause viscous blockage in distant smaller blood vessels. Such blockage can result in cerebral vascular strokes and failure of other vital organs such as the kidneys. Including the fluid or peri-bellows fluid within space 212 of housing 210 and within housing 122 (which can, for example, be in fluid connection with housing 210) to bathe components of motor 120, speed reducer 160 and converter 180, eliminates gases. Moreover, the fluid can operate to dissipate heat from pump system 100. Motion of valve assembly 300 results in circulation or movement of the bathing fluid within pump system 100 and heat can be dissipated, for example, to the blood stream via flow conduit 240.

A lubricant, which can, for example, be a biocompatible, aqueous or hydrophilic lubricant can, for example, be included in the bathing fluid. An example of such a fluid is the glycosaminoglycan hyaluronic acid, which occurs naturally in the body.

As valve assembly 300 moves forward (that is, in the direction of blood flow from left ventricle 14 of heart 10), one or more openings or ports thereof are closed and valve assembly 300 drives blood forward toward the upper portion of ascending aorta 22. The motion of valve assembly 300 is then reversed and it's port(s) are opened, allowing the momentum of the blood to continue forward blood flow.

In the illustrated embodiment, valve assembly 300 includes a single port 320 having a generally circular shape (see, for example, FIG. 3A) and is closed or opened via one or a plurality of movable closure members 330 (two in the illustrated embodiment). Port 320 can, for example, have a diameter approximately equal to the diameter of the ascending aorta. Closure members 330 can, for example, rotate to a closed position (see, FIG. 4B) and to a range of open positions (see, FIGS. 3A and 4C) via shafts or rods 332. In the fully open position, closure members 330 can, for example, be oriented substantially parallel to flow to reduce resistance and to reduce the potential blood strain/shear. Pressure from blood within flow conduit 240 can, for example, be used to open and close closure members 330. If, for example, power to pump system 100 fails or pump system 100 otherwise malfunctions, closure members 330 can still be opened by blood flow from the heart. In that regard, as the closure members 330 can be designed to require only a few millimeters of mercury or less increased pressure to open and pump blood therethrough, blood is free to flow through pump system 100 even if pump system 100 is inoperable.

The distance traveled in any one direction by valve assembly 300 can, for example, be in the range of approximately 1 to 2 centimeters. The cross sectional area of the blood contacting surface of valve assembly 300 can, for example, be approximately 10 square centimeters. As one example, the valve stroke of such a valve assembly can be 1.5 centimeters, resulting in a displaced volume of 15 milliliters. It has been found by experimentation that at cycle rates between, for example, 10 and 16 cycles per second, an aqueous fluid will flow continuously forward because of a momentum effect even though roughly half the time valve assembly 300 is moving backwards. For example, three cycles of 1.5 centimeter valve movement of the above-described valve assembly displaces roughly 3 times 15 or 45 milliliters of blood, and the actual flow in the forward direction could be the same or even greater than this amount. This output provide sufficient extra flow to compensate for the low cardiac output found in typical heart failure. In a number of embodiments, valve thickness and end ring thicknesses are kept below approximately 70 mils (1.78 mm) and approximately 100 mils (2.54 mm), respectively, to facilitate stroke lengths and flow assist volumes as described above.

In the illustrated embodiment, a valve assembly carriage or bearing assembly 400 (see, for example, FIGS. 6A through 6D), which can, for example, include linear rolling element bearings 405, is provided to constrain and align the reciprocating (or forward and backward) motion of valve assembly 300. Valve peripheral support structure or valve ring 310 can, for example, be operatively connected to or captured by an annular connector 410 of bearing assembly 400. Annular connector 410 is operatively connected to rotary-to-linear converter 180 so that annular connector 410 (and thereby valve assembly 300) is driven in a reciprocating linear manner by drive system 110.

Annular connector 410 can, for example, be operatively connected to a plurality of generally linear rolling element bearings 405 (three in the illustrated embodiment). See, for example, FIGS. 6A through 6E. Each linear rolling element bearing 405 includes an inner race member 420 (which can be connected to or formed integrally or monolithically with annular connector 410) and an outer race member 450. As illustrated in FIG. 6D, each of inner (relative to axis A₁) race members 420, can include one or more races 430. In the illustrated embodiment, linear races 430 include a central, gear or toothed section 432. On each side of gear section 432 are bearing surfaces 434 which can, for example, be angled or tapered. In the embodiment illustrated in FIG. 6D and 6E, a dual taper is provided wherein the bearing surface 434 first tapers inward (with respect to axis A₁) as it extends from gear section 432 and then outward. As set forth above, for each race member 420 of linear rolling element bearings 405, a corresponding outer race member 450 (relative to axis A₁) is provided. Race members 450 are seated in or fixed to end members or rings 242 and 244 (to which conduit 240 can be attached by, for example, an adhesive). End members 242 and 244 can, for example, be fixed in position relative to each other via race members 450 and/or operative connection with housing 210. Race members 450 include one or more linear races (not shown) which are generally identical to races 430.

Roller elements or rollers 460 are operatively connected between races 430 of inner race members 420 and the races of outer race members 450. Roller elements 460 include a central gear or toothed section 462 and bearing surfaces 464 on each side thereof Bearing surfaces 464 are angled, tapered or canted in the illustrated embodiment to include a dual taper corresponding to and mating with dual tapered bearing surfaces 434 of races 430 and the dual tapered bearing surfaces (not shown) of the races of outer race members 450.

As annular connector 410 is driven in a linear reciprocating manner to drive valve assembly 300, roller elements 460 roll along races 430 of inner race members 420 and along the races of outer race members 450. The relative positions of roller bearing elements 460 on a race are fixed by the cooperation or intermeshing of central gear section 462 and the gear sections of the bordering races. As described above for rolling element bearing 130, cages are not required between roller elements 460.

In the embodiment illustrated, for example, in FIGS. 6A through 6E rotary motion from ring gear 164 of speed reducer 160 (which can, for example, reduce the rotational speed of rotary motor 120 by 3 to 8 times) is converted to linear motion, for example, using a post or extending member 182 in eccentric connection with ring gear 164. A rotating member 190 (in the form, for example, of a rolling element bearing) is connected to post 182 via a passage 192 therein. Rotating member 190 can, for example, include a radially inward positioned gear wheel 193, a radially outward positioned, rotating ring 194 including a race 194a, and a plurality of rolling elements 195 therebetween. Rotating member 190 engages and follows a cam element or surface 198 of a cam member 196 which is connected to annular connector 410 (and thereby to valve assembly 300) to move valve assembly 300 in a linear reciprocating manner. As described above, linear motion of valve assembly 300 is supported and aligned by linear rolling element bearings 405. Aspects of the linear reciprocating motion of valve assembly 300 can be adjusted by variance of cam element 198. For example, valve assembly 300 can be returned to its rearwardmost position during the backward with a different velocity profile than the velocity profile of the forward stroke.

FIG. 6D illustrates another system or mechanism for translating rotary motion to the linear, reciprocating motion of valve assembly 300. A connecting arm or crank 190′, which includes rolling element bearings 195′ at each end thereof (as described in connection with rotating member 190), is connected to post 182 at one end thereof and to annular connector 410 at the other end thereof. Connecting arm 190′ provides translation of the rotary motion of speed reducer 180 to linear motion. In this type of rotary-to-linear translation or conversion, the amount of rotation of ring gear 164 required for a forward stroke of valve assembly 300 can be different from the amount of rotation of ring gear 164 for a rearward stoke of valve assembly 300. For example, in one embodiment, greater than half of a full ring gear rotation cycle is used for driving the forward stroke of valve assembly 300. By offsetting the position of the bearing of ring gear 164 from the linear direction of valve assembly movement, the ratio of this unequal translation can be varied. Converters for converting the rotary or rotational motion of rotary motor 120 to a nonlinear or curvilinear, reciprocating motion can be provided if it is desired to drive valve assembly 300 in a nonlinear or curvilinear, reciprocating manner.

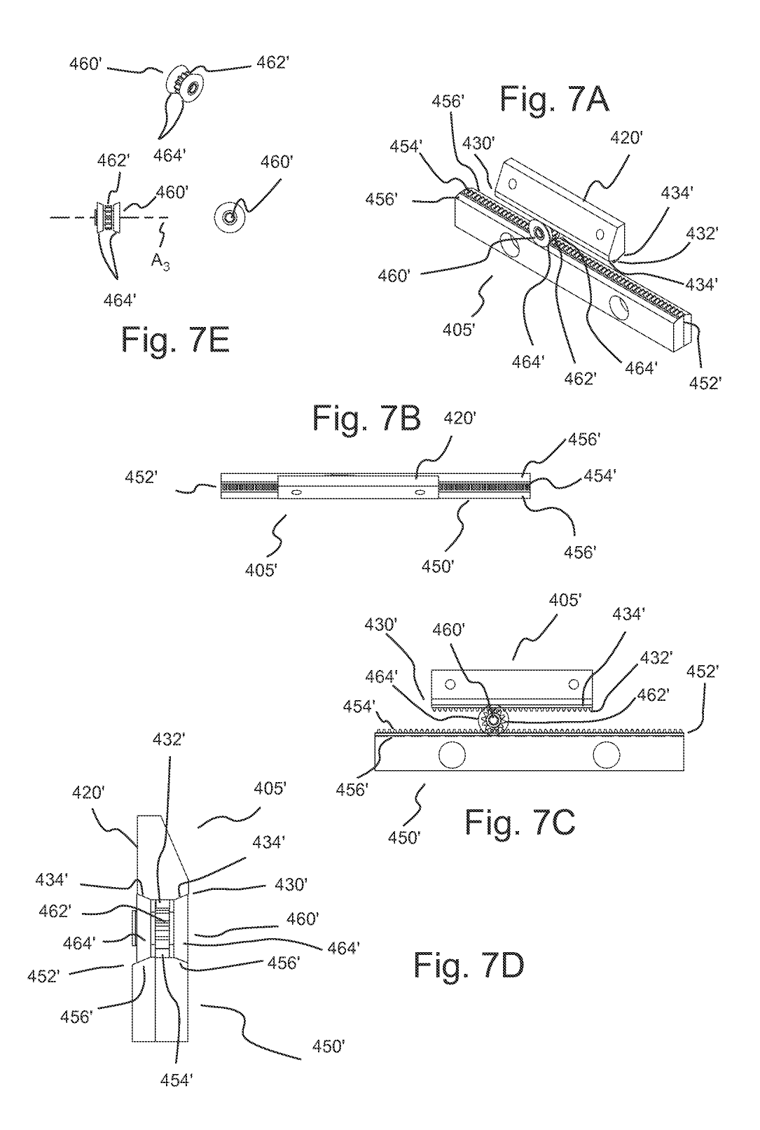

FIGS. 7A through 7E illustrate an alternative embodiment of a linear rolling element bearing 405′ that can, for example, be used in connection with bearing assembly 400. A first race member 420′ includes a linear race 430′. In the illustrated embodiment, linear race 430′ includes a central, gear or toothed section 432′. On each side of gear section 432′ are bearing surfaces 434′, which are angled or tapered. A second race member 450′ includes a linear race 452′ including a central, gear or toothed section 454′. On each side of gear section 454′ are bearing surfaces 456′, which are angled or tapered. One or more geared rolling elements 460′ are positioned between first race member 430 and second race member 450 so that a gear section 462′ of roller elements 460′ intermeshes with gear sections 432′ and 454′ of races 430′ and 452′. Angled or tapered bearing surfaces 464′ extend laterally outwardly from each side of gear section 462′ of roller elements 460′. In the illustrated embodiment of FIGS. 7A through 7E, bearing surface 464′ angle or taper radially outward (relative to axis A₃ thereof—see FIG. 7E) as they extend away from gear section 462′.

The geared roller elements of the rolling element bearings described above each include an intermediate or central gear section from which tapered bearing surfaces extend. However, roller elements including two geared sections having an intermediate roller bearing surface extending therebetween can be used. The intermediate roller bearing surface can, for example, have a generally circular cross section that changes in diameter over the length thereof to provide an angled, tapered or canted bearing surface. As also described above, other types of bearings, and particularly rolling element bearings, including ball bearings with races can be suitable for at least some mechanical linkages of the pump systems hereof.

Pump lifetime reliability considerations described above are complicated when the fluid surrounding flow conduit or bellows 240 also bathes the mechanical motor to valve assembly linkage. Once again, this fluid should be compatible with blood so that if any leakage of this fluid should occur into the blood stream, such peri-bellows fluid would not result in significant or any injury to the patient. As described above, oily or hydrophobic lubricant fluid, normally used with mechanical linkages having multiple bearings would be injurious if released. A truly blood compatible fluid can, for example, include dissolved sodium chloride and possibly other salts in similar concentrations as found in the blood. However, such salt solutions are quite corrosive when placed in contact with virtually all bearing hard steels, including so-called bearing hard 440 series stainless steels. In a number of embodiments hereof, bearing-hard, nitrided martensitic stainless steel (for example, CRONIDUR R 30™, available from Energietechnik Essen GmgH of Essen, Germany) that is corrosion resistant is used in the bearings or rolling element bearings of pump system 100. See German Patent No. DE3901470, the disclosure of which is incorporated herein by reference. Bearings including races and/or rolling elements formed from CRONIDUR are, for example, available from The Barden Corporation. Further, bearings having races and/or rolling elements made from a hard ceramic material can be used to provide corrosion resistance and suitable lifetime requirements.

In addition to the risk of bearing corrosion, there is also a risk of corrosion of copper wire conductors of rotary motor stator 124. Corrosion of such wires when exposed to the corrosive salt water environment of the fluid within housing sections 122 and 210 poses a failure mode for pump system 100. To lessen or eliminate this risk, stator 124 can, for example, be sealed in a hermetically welded titanium case using feedthroughs and sealed crimp joints to connect the copper wires of stator 124 with corrosive resistant conductors such as, for example, platinum wire in the feedthroughs and DFT® silver filled stainless tubing conductors in the leads exiting the motor stator hermetically sealed case. DFT® wire is a metal-to-metal composite available from Fort Wayne Metals of Fort Wayne, Indiana.

Using, for example, a three dimensional depiction of the human anatomy surrounding ascending aorta 22, as found, for example, in the commercially available Visual Human software produced by the University of Washington Medical School, one can analyze the dimensions of the various organs in this peri-aortic space. In the case of pump system 100, the volume of drive system 110 (including, rotary motor 120, speed reducer 160 and rotary-to-linear convertor 180) can fit within the volume of lung space without significantly impinging on the other critical structures. As described above, the components of drive system 110 can, for example, be generally cylindrical in shape and extend into the right lung space in a right anterior oblique direction from the aorta at approximately a right angle from flow conduit assembly 200, which can also be generally cylindrical in shape. As described above, the displacement volume of drive system 110 may be made as small as, for example, less than 150 cc, less than 100 cc, or even less than 50 cc. As also described above, distance D₁ from axis A₁ to the distal end of housing section 122 of drive system 110 may, for example, be less than 12 cm, less than 10 cm or even less than 8 cm. The amount of sacrificed lung volume arising from pump system 100 should not significantly affect lung function, given, for example, an average right lung volume of 3200 cc.

Compared with currently available implantable heart assist pump systems, system 100 affords substantial functional improvements and minimally impacts upon surrounding organs and their function (and, particularly, minimally impacts lung function).

The foregoing description and accompanying drawings set forth a number of representative embodiments at the present time. Various modifications, additions and alternative designs will, of course, become apparent to those skilled in the art in light of the foregoing teachings without departing from the scope hereof, which is indicated by the following claims rather than by the foregoing description. All changes and variations that fall within the meaning and range of equivalency of the claims are to be embraced within their scope.

What is claimed is:

1. An implantable pump system for assisting blood flow, comprising:
a flexible conduit;
at least one movable valve in fluid connection with the flexible conduit;
a drive system comprising a rotary motor and a speed reducer operatively connected to the rotary motor; and
a converter operatively connected to the drive system and operatively connected to the valve to drive the valve in a reciprocating and approximately linear manner.

2. The pump system of claim 1 wherein the speed reducer comprises a spur gear driving a ring gear, and wherein the converter is operatively connected to the speed reducer.

3. The pump system of claim 2 wherein the ring gear is in operative connection with the converter.

4. The pump system of claim 3 wherein the converter comprises an eccentric member extending from the ring gear.

5. The pump system of claim 4 wherein the converter further comprises a rotating element connected to the eccentric member that engages a cam member operatively connected to the valve to drive the valve in a reciprocating, linear manner.

6. The pump system of claim 1 wherein any moving mechanical linkage of the drive system and between the drive system and the valve comprises at least one rolling element bearing resistant to corrosion by a salt solution.

7. The pump system of claim 6 wherein the rolling element bearing is constructed from a ceramic material or from a nitrided hardened stainless steel material.

8. A pump system of claim 1 wherein the flexible conduit is positioned within a sealed housing, a volume between the flexible conduit and housing being filled with an aqueous fluid having dissolved solutes to provide an osmolarity approximately equal to the osmolarity of blood.

9. The pump system of claim 8 wherein the fluid is an aqueous salt solution.

10. The pump system of claim 9 wherein the drive system is in fluid connection with the volume and the fluid is present within the drive system.

11. The pump system of claim 10 wherein the fluid is adapted to dissipate heat from the drive system.

12. The pump system of claim 10 wherein the fluid comprises at least one hydrophilic lubricant.

13. A pump system of claim 1 wherein the flexible conduit is adapted to be placed in series with the aorta.

14. A pump system of claim 1 wherein the flexible conduit is adapted to be placed in line with the aorta.

15. A method of assisting blood flow, comprising: implanting a pump system within a body, the pump system comprising a flexible conduit, at least one moveable valve in fluid connection with the flexible conduit, a drive system comprising a rotary motor and a speed reducer operatively connected to the rotary motor, and a converter operatively connected to the drive system and to the valve to drive the valve in a reciprocating linear manner.

16. A pump system for assisting blood flow, comprising:
a flexible conduit;
at least one movable valve in fluid connection with the flexible conduit; and
a sealed housing encompassing at least a portion of the flexible conduit, a volume between the housing and the flexible conduit comprising an aqueous fluid having an osmolarity approximately equal to the osmolarity of blood.

17. The pump system of claim 16 wherein the fluid is an aqueous salt solution.

18. The pump system of claim 17 wherein the drive system is in fluid connection with the volume and the fluid is present within the drive system.

19. The pump system of claim 18 wherein the fluid is adapted to dissipate heat from the drive system.

20. The pump system of claim 18 wherein the fluid comprises at least one hydrophilic lubricant.

21. The pump system of claim 16 further comprising a drive system comprising a rotary motor and a speed reducer operatively connected to the rotary motor; and a converter operatively connected to the drive system and operatively connected to the valve to drive the valve in a reciprocating and approximately linear manner.
